(19) United States
(12) Patent Application Publication
(10) Pub. No.: US 2026/0207261 A1
(43) Pub. Date: Jul. 23, 2026

(54) FLEXIBLE DEVICE LENGTH ESTIMATION FROM MOVING FLUOROSCOPY IMAGES

(71) Applicant: KONINKLIJKE PHILIPS N.V., EINDHOVEN (NL)

(72) Inventors: Ayushi SINHA, CLEVELAND HEIGHTS, OH (US); Leili SALEHI, WALTHAM, MA (US); Javad FOTOUHI, CAMBRIDGE, MA (US); Vipul Shrihari PAI RAIKAR, SOMERVILLE, MA (US); Ramon Quido ERKAMP, SWAMPSCOTT, MA (US)

(21) Appl. No.: 19/135,862

(22) PCT Filed: Dec. 4, 2023
(86) PCT No.: PCT/EP2023/084032

§ 371 Date: Jun. 5, 2025

Related U.S. Application Data
(60) Provisional application No. 63/431,759, filed on Dec. 12, 2022.

(51) Int. Cl. A61B 34/20; A61B 90/00; G06T 7/00; G06T 7/246; G06T 7/60; G06T 11/00; G06V 10/26; G06V 10/70; G06V 20/40; G06V 20/50

(52) U.S. Cl. CPC A61B 34/20; G06T 7/0016; G06T 7/248; G06T 7/60; G06T 11/00; G06V 10/26; G06V 10/70; G06V 20/41; G06V 20/50; A61B 2034/2065; A61B 2090/061; A61B 2090/376; G06T 2207/10016; G06T 2207/10064; G06T 2207/20081; G06T 2207/30021; G06T 2210/41; G06V 2201/034

(57) ABSTRACT

A device (10) for determining an inserted length of an interventional instrument. The device includes a processor (20) configured to receive imaging data (35) comprising an anatomy within a patient, identify, from the imaging data, a portion of the interventional instrument (12) inserted into the patient and disposed within the anatomy, and predict the inserted length of the interventional instrument based on the identified portion of the interventional instrument.

### FIELD

The following relates generally to the endovascular arts, device selection arts, artificial intelligence (AI) arts, and related arts.

### BACKGROUND

Using endovascular devices of appropriate length for each patient is important in ensuring the best outcome for the patient. As an example, catheter lengths may vary for patients of different height, depending on the application the catheter is being used for. During central venous catheter (CVC) placement, for instance, improper catheter length can increase the risk of catheter migration or displacement (see, e.g., Roldan, C. J., & Paniagua, L. (2015). Central Venous Catheter Intravascular Malpositioning: Causes, Prevention, Diagnosis, and Correction. The western journal of emergency medicine, 16(5), 658-664.https://doi.org/10.5811/westjem.2015.7.26248) and may require additional procedures to reposition the catheter and prevent vascular complications.

Even if improper catheter length is recognized before the end of the procedure, additional procedure time is required to extract and insert a catheter of appropriate length. Procedures using endovascular robots could require even more time for device replacement since the old device would need to be removed from the robot, and a new device inserted into the robot before inserting into patient vasculature. Increased procedure time can increase the risk of complications, and use of multiple catheters increases waste and adds cost.

The following discloses certain improvements to overcome these problems and others.

### SUMMARY

In some embodiments disclosed herein, a system for determining an inserted length of an interventional instrument includes a processor in communication with memory. The processor is configured to receive imaging data comprising an anatomy within a patient, identify, from the imaging data, a portion of the interventional instrument inserted into the patient and disposed within the anatomy, obtain scaling information associated with the imaging data, and predict the inserted length of the interventional instrument based on the identified portion of the interventional instrument and the scaling information.

In some embodiments disclosed herein, a non-transitory computer readable medium stores instructions which, when executed by a processor, cause the processor to receive imaging data comprising an anatomy within a patient, identify, from the imaging data, a portion of the interventional instrument inserted into the patient and disposed within the anatomy, obtain scaling information associated with the imaging data, and predict the inserted length of the interventional instrument based on the identified portion of the interventional instrument and the scaling information.

In some embodiments disclosed herein, a method of determining an inserted length of an interventional instrument. The method includes receiving imaging data comprising an anatomy within a patient, identifying, from the imaging data, a portion of the interventional instrument inserted into the patient and disposed within the anatomy, obtaining scaling information associated with the imaging data, and predicting the inserted length of the interventional instrument based on the identified portion of the interventional instrument and the scaling information.

One advantage resides in reducing delays and costs during endovascular procedures.

Another advantage resides in determining an inserted length of an endovascular device during an endovascular procedure.

Another advantage resides in determining the inserted length of an endovascular device in real-time during an endovascular procedure.

Another advantage resides in determining the inserted length of an endovascular device during an endovascular procedure based on medical imaging usually performed to provide image guidance to the surgeon during the procedure.

Another advantage resides in using imaging in determining both an inserted length of an endovascular device for an endovascular procedure and a confidence value for the determined inserted length.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an endovascular device in accordance with the present disclosure.

FIG. 2 diagrammatically illustrates a method of performing a vascular therapy method using the device of FIG. 1.

FIG. 3 diagrammatically shows operation of a model receiving as input images depicting a portion of anatomy of a patient and a portion of an associated interventional instrument that is inside the patient, and outputs an estimated length of the interventional instrument that is inserted into the anatomy of the patient.

FIG. 4 diagrammatically shows a visualization displayed by the device of FIG. 1 where a field of view of the imaging device change resulting in a displacement in the background and a where a device is inserted into the anatomy of the patient resulting in a displacement of the device position.

FIG. 5 shows another embodiment of operations of the NN of FIG. 3.

### DETAILED DESCRIPTION

During an intravascular procedure in which a catheter or other interventional instrument is inserted into the vasculature of a patient, the insertion is commonly visualized using fluoroscope imaging or another suitable imaging modality. Such procedures are sometimes referred to by nomenclatures such as image-guided therapy (IGT). The image guidance may be performed in real-time, e.g., as a time sequence of images to provide a CINE view of the procedure. The image guidance may provide the surgeon or other person performing the procedure with visual guidance in real-time as to the current location of the interventional instrument (e.g., tip of the interventional instrument) as the interventional instrument is inserted into the patient, as well as information about the surrounding vasculature and/or other tissue or organs.

In embodiments disclosed herein, imaging such as that used to provide visual image guidance to the operator is advantageously also used to estimate and provide the inserted length of the interventional instrument in real-time. The inserted length is the length of the interventional instrument that is currently inserted into the patient. The inserted length of the interventional instrument may comprise all or a portion of the entire length of the interventional instrument. Such inserted length estimation based on a time sequence of images is challenging. The operator may adjust the location of the imaging field of view (FOV) to follow the tip of the interventional instrument as it progresses through the body. Hence, the time series of images may include both motion of the interventional imaging device and motion of the background. These motions may in general be independent. Moreover, the FOV will usually only encompass a distal portion of the interventional instrument. Still further, the interventional images may be two-dimensional (2D) images, for example, fluoroscopy images acquired using a flat detector plate, further complicating extraction of the length of the inserted portion of a device in three-dimensional (3D) space from 2D images.

Recent work in machine learning and computer vision has explored methods to estimate features from moving objects by separating the target object from its background (see, e.g., Zhengqi Li, Tali Dekel, Forrester Cole, Richard Tucker, Noah Snavely, Ce Liu & William T. Freeman. (2019). Learning the Depths of Moving People by Watching Frozen People. CVPR.). However, these methods have been developed for natural world data. For instance, one application predicts the depth of a target (e.g., a person) in a sequence of images with moving or changing background (e.g., camera moving with the person). In this application, the target and background depths are changing at different rates. Since the camera is moving with the person, the depth estimates of the person's arms and legs may only change to accommodate their walking, while the depth estimates of the background may change more drastically. Natural world images, however, contain more features and information than medical images. Some embodiments for estimating the inserted length of an interventional instrument adapt such techniques to this different task.

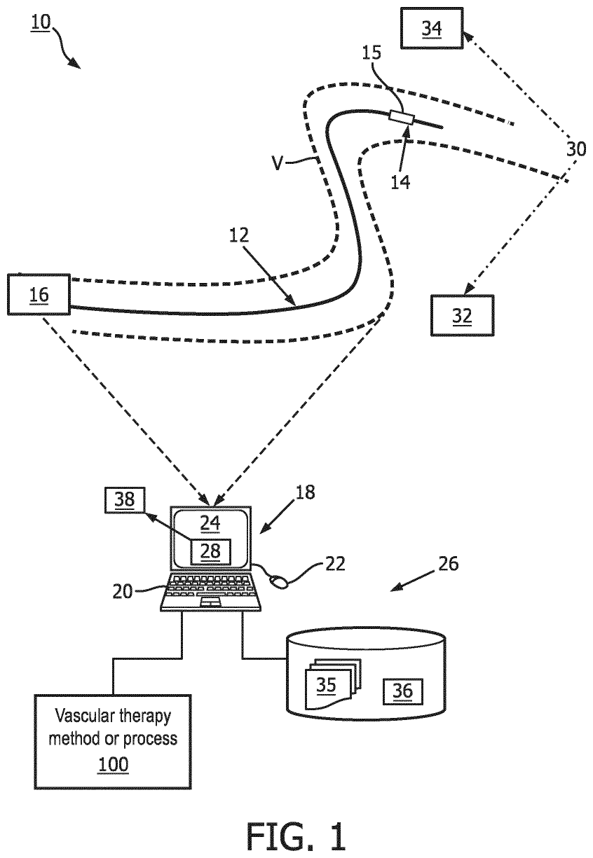

With reference to FIG. 1, a system 10 is diagrammatically shown. The system 10 may include, for example, an endovascular device, an endobronchial device, a surgical device (e.g., needle), or any other suitable device. As shown in FIG. 1, the system 10 includes an interventional device or instrument 12 (e.g., a catheter, a guidewire, and so forth—diagrammatically shown in FIG. 1 as a line) configured for insertion into a portion of anatomy of a patient, such as into a blood vessel V containing a target such as an occlusion or a clot or so forth. As seen in FIG. 1, the interventional device or instrument 12 is flexible so that it can follow the contours of the blood vessel V as it is inserted. In a typical intravascular or endovascular procedure, the surgeon or other operator accesses a target by creating an incision (not shown) and inserting a tip 14 of the interventional instrument 12 into a blood vessel V via the incision, and then pushing the interventional instrument 12 into and through the blood vessel V until the tip 14 reaches the target. The interventional instrument 12 is generally radiopaque at least to the extent that it is visible (potentially with low contrast) in X-ray imaging. The interventional instrument 12 optionally includes a tip element 15 located at its tip 14 that is highly radiopaque, so that the tip 14 of the interventional instrument 12 is more easily imaged in fluoroscopic imaging. For example, the tip element 15 located at the 14 of the interventional instrument 12 may be a coating of a radiopaque material disposed on the tip 14, or may comprise an attached radiopaque ring 15 made of, for example, platinum or Nitinol wire that is metallurgically bonded (e.g., by welding) to the tip 14 of the interventional instrument 12. These are merely illustrative examples.

In an example embodiment, a clinician controls movement of the interventional instrument 12 through the blood vessel V; however, the movement of the interventional instrument 12 can also be robotically controlled. FIG. 1 also shows a robot 16 (diagrammatically shown in FIG. 1 as a box) operatively connected to the proximal end of the interventional instrument 12, that is, to the end opposite from the tip 14. The robot 16 (and more generally a proximal portion of the interventional instrument 12) is located outside of the patient, and more particularly outside of the blood vessel V. As the interventional instrument 12 is pushed into the blood vessel V (either manually or robotically), the length of the interventional instrument 12 that is disposed inside the blood vessel V increases. The optional robot 16 is configured to control movement of the interventional instrument 12 into and through the blood vessel V. Robotic control may be performed by the clinician using controllers such as a joystick or mouse clicks on a user interface or may be performed automatically using an autonomous control system that can steer the robot 16.

FIG. 1 further shows a hardware processing device 18, such as a workstation computer, a smart tablet, or more generally a computer which can be used to control the robot 16 to automatically perform the insertion and movement of the interventional instrument 12 through the vessel V. The processing device 18 may also include a server computer or a plurality of server computers, e.g., interconnected to form a server cluster, cloud computing resource, or so forth, to perform more complex computational tasks. The electronic processing device 18 includes typical components, such as a hardware processor 20 (e.g., a microprocessor), at least one user input device (e.g., a mouse, a keyboard, a trackball, and/or the like) 22, and a display device 24 (e.g., an LCD display, plasma display, cathode ray tube display, and/or so forth). In some embodiments, the display device 24 can be a separate component from the processing device 18 or may include two or more display devices.

The processor 20 is operatively connected with one or more non-transitory storage media 26. The non-transitory storage media 26 may, by way of non-limiting illustrative example, include one or more of a magnetic disk, RAID, or other magnetic storage medium; a solid-state drive, flash drive, electronically erasable read-only memory (EEROM) or other electronic memory; an optical disk or other optical storage; various combinations thereof; or so forth; and may be for example a network storage, an internal hard drive of the electronic processing device 18, various combinations thereof, or so forth. It is to be understood that any reference to a non-transitory medium or media 26 herein is to be broadly construed as encompassing a single medium or multiple media of the same or different types. Likewise, the processor 20 may be embodied as a single processor or as two or more processors. The non-transitory storage media 26 stores instructions executable by the at least one processor 20. The instructions include instructions to generate a visualization of a graphical user interface (GUI) 28 for display on the display device 24.

FIG. 1 also diagrammatically shows an imaging device 30 configured to acquire single-shot images and/or a time sequence of images or imaging frames 35 of a position, or movement, of the interventional instrument 12 (e.g., a distal portion of the instrument 12 including the radiopaque tip 14). In the illustrative examples, the imaging device 30 is a fluoroscopic imaging device (e.g., an X-ray imaging device, C-arm imaging device, a CT scanner, or so forth) and the interventional instrument 12 is visible under the fluoroscopic imaging. The fluoroscopic imaging is in some embodiments real-time imaging, e.g., with images being acquired at a frame rate of 15-60 frames/second (i.e., 15-60 fps) in some nonlimiting illustrative embodiments. The imaging device 30 may be in communication with the at least one processor 20 of the processing device 18. As shown in FIG. 1, the imaging device 30 comprises an X-ray imaging device including an X-ray source 32 and an X-ray detector 34, such as a C-arm imaging device; however, it will be appreciated that any suitable imaging device, such as ultrasound (US), computed tomography (CT), flat-panel X-ray or fluoroscope, magnetic resonance imaging (MRI), or any other suitable imaging device may be used. It should be noted that FIG. 1 illustrates the X-ray source 32 and X-ray detector 34 diagrammatically-in practice the field of view of the imaging device 30 should be large enough to encompass at least a distal portion of the interventional instrument 12, which may include the tip 14 of the interventional instrument 12, and the surrounding vasculature and/or anatomy. The images 35 can be stored in the non-transitory storage media 26.

The at least one processor 20 is configured as described above to perform process 100, which may be a vascular diagnosis method, a vascular therapy method, an endobronchial diagnosis method, an endobronchial therapy method, a surgical method, etc. The non-transitory storage medium 26 stores instructions which are readable and executable by the at least one processor 20 to perform disclosed operations including performing, for example, a vascular therapy method, or process 100. In some examples, the method 100 may be performed at least in part by cloud processing.

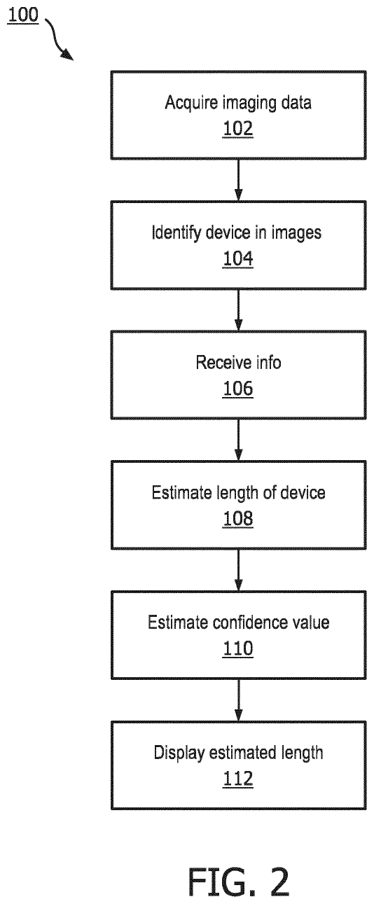

Referring to FIG. 2, and with continuing reference to FIG. 1, is shown an illustrative embodiment of method 100 as a flowchart. To begin the method 100, the interventional instrument 12 is inserted into the blood vessel V and pushed into the vasculature using the robot 16 or manually.

At operation 102, the imaging device 30 acquires the time sequence of images 35 (or imaging frames) of the patient. The received time sequence of images 35 may comprise two-dimensional (2D) imaging data that includes a portion of the interventional instrument 12 and the portion of anatomy of the patient in which the portion of the interventional instrument is disposed (e.g., the vessel V). The imaging operation 102 may be performed during the interventional procedure to provide visual guidance to the physician as the physician manipulates the proximal end of the interventional instrument 12 to push the instrument 12 through the vasculature toward a clot or other target within the patient. The imaging device 30 communicates the time sequence of images 35 to the processing device 18.

At operation 104, the processing device 18 identifies the portion of the interventional instrument 12 in the sequence of images 35. In embodiments, all or part of the portion of the interventional instrument is not visible or detectable in the sequence of images by a user (e.g., physician) of the system 10. The identification may be performed by automated segmentation based on a priori knowledge of the expected appearance of the interventional instrument 12 in the images, such as the expected width of the instrument image based on its known diameter and imaging characteristics, an expected grayscale intensity range for the image pixels representing the interventional instrument 12 in the image (for example, based on the radiodensity of the interventional instrument 12 on the Hounsfield scale, in the case of X-ray imaging), a priori knowledge of the maximum bend radius of the interventional instrument 12, and/or so forth.

At operation 106, the processing device 18 obtains scaling information indicative of scaling associated with the images 35. The processing device may determine the length of the interventional instrument 12 inserted into the patient based on the images 35 and the scaling information. In some embodiments, the received scaling information includes one or more of an amount of movement of a component of the system 10, patient size information, a three-dimensional (3D) image of the portion of anatomy of a patient in which the interventional instrument 12 is disposed, and information of dimensions of the interventional instrument 12 (e.g., length, size (e.g., 6 French), a distance between the tip element 15 and the tip 14, flexibility, and so forth).

At operation 108, the processing device 18 determines or estimates or predicts the inserted length of the interventional instrument 12 from the time sequence of images 35. In some embodiments, only a portion of the interventional instrument is currently inserted into the anatomy of the patient and the inserted length is the length of the currently inserted portion of the interventional device. In embodiments, the inserted length may comprise all or a portion of the entire length of the interventional instrument. In some embodiments, the inserted length may be determined based on features (e.g., shape, dimensions, position, components on instrument, etc.) of the portion of the interventional instrument extracted from the images and features of the surrounding anatomy (e.g., landmarks, shape, dimensions, location within body, etc.) extracted from the images, such as extracted by image feature extraction techniques known in the art. In some embodiments, the inserted length may also be determined based on the scaling information of operation 106. In an example embodiment, the processing device 18 may compute the length of the portion of the interventional instrument in the images based on the extracted features of the portion and the surrounding anatomy, and then, apply the scaling information to the computed length of the portion of the interventional device in the images to compute the inserted length of the interventional instrument.

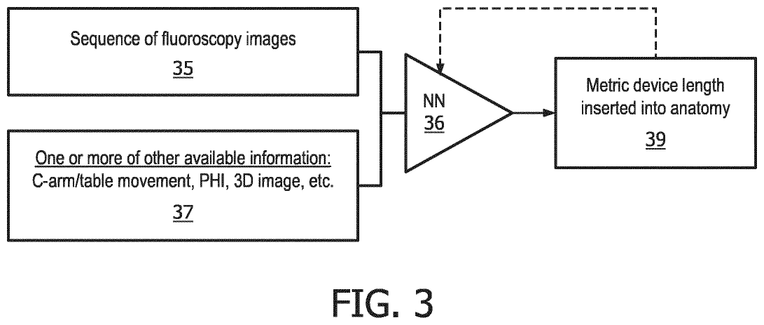

In some embodiments, the determination of the inserted length of the interventional instrument 12 may be performed by implementing a model 36, such as a machine learning (ML) model which may be based on hand-crafted feature extractors. For instance, the model may comprise relevant image parameters or features extracted by feature extractors applied to Gaussian Mixture Models, Expectation Maximization, Hidden Markov Models, etc. In some embodiments, the model may be based on features learned using a neural network (NN). With brief reference to FIG. 3, in some embodiments, the processing device 18 may perform the inserted length determination by applying the model 36 configured as an artificial neural network (ANN) 36 stored in the non-transitory storage medium 26 of the processing device 18. The ANN 36 may have been previously trained from historical data, such as historical imaging data and patient data (for example, endovascular imaging data and endovascular patient data), to determine the inserted length of the interventional instrument 12 based on the current portion of an interventional instrument present in a current image and the current portion of the anatomy present in the current image. In some embodiments, the historical data may include historical scaling information and the ANN has been trained to also determine the inserted length based on current scaling information.

As diagrammatically shown in FIG. 3, the ANN 36 may receive as input a time sequence of images 35, and other data (indicated as element 38) including, for example, patient health information (e.g., patient height, patient age, etc.), a preoperative or intraoperative three-dimensional (3D) image of the patient (including a portion of the interventional instrument 12), an amount of C-arm movement, an amount of patient table movement, or any other information that allows scaling an estimated device length from the images 35 to metric length. The ANN 36 may then predict and output the metric length of the device inserted into the patient's anatomy (indicated as element 39) based on the input data. In some embodiments, the ANN 36 may be trained with the time sequence of images 35. Training of the ANN 36 may include tuning ANN parameters comprising model weights and biases using training data (e.g., image data, scaling data, etc. from previous procedures) such that the trained ANN 36 accurately predicts the expected output data from new input data.

Referring back to FIG. 2, in some embodiments, the processing device 18 (operation 108) determines the inserted length of the interventional instrument 12 based on analysis of the portion of the interventional instrument 12 present in the images 35 and one or more anatomical landmarks present in the images 35. In some embodiments, the processing device 18 applies an artificial neural network (ANN) 36 that is trained to determine such inserted length using image features extracted from the images, including features of the portion of the interventional instrument 12 present in the images 35 and the one or more anatomical landmarks present in the images 35. In some embodiments, there may not be a continuous stream of fluoroscopy images 35 from the access site to the current location of the interventional instrument 12 in the anatomy. In some situations, navigation of the interventional instrument 12 near the access site is not performed under fluoroscopy, and fluoroscopy is only initiated near more complex vasculature. In other situations, fluoroscopy may be used near the access site, but not used in a subsequent section, and then reinitiated near more complex vasculature. In such situations, data is missing from the acquired images 35 and the ANN 36 must infer the inserted length of the interventional instrument 12 from background features that inform which part of the anatomy is currently being imaged. For instance, image features in the background, along with patient height, can allow the processing device 18 to estimate a metric length of the inserted length of the interventional instrument 12 even if prior fluoroscopy sequences are not available.

In some embodiments, the at least one image of the imaging data 35 includes at least two images depicting different views of the portion of the interventional instrument 12 present in the images and the one or more anatomical landmarks. In this embodiment, if multiple C-arm views from the same time are available (e.g., data acquired from a biplane system), the processing device 18 uses the data from the multiple views to compute a more precise estimate of the length of the interventional instrument 12 currently inserted into the patient since this data would provide more information about the interventional instrument 12 and the background and, hence, increase the confidence of the predictions (e.g., reduce ambiguities from foreshortening) by the processing device 18. The multiple views may be inputted as separate input channels into the ANN 36 or as separate inputs into a Siamese network architecture, where parallel convolutional layers process the multiple views separately in the early layers of the ANN 36, and merge the ANN 36 weights in later layers to provide a combined output.

In some embodiments, the processing device 18 (operation 108) keeps track of the inserted device length estimates from previous images (or imaging frames) 35 to generate consistent outputs as the interventional instrument 12 is inserted into the patient. Such tracking may be performed as a post-processing step to generate smooth outputs of estimated device length by, for instance, outputting the average of a set of most recent device length predictions from consecutive imaging frames 35. Alternatively, the ANN 36 may use its most recent output or a set of most recent outputs as an input to predict the subsequent length of the interventional instrument 12 inserted into the patient (as indicated by a dotted arrow in FIG. 3).

In some embodiments, the processing device 18 (operation 108) determines the length of the interventional instrument 12 currently inserted into the patient as an accumulation of incremental changes in the inserted length in successive images of the time sequence of images 35. In some embodiments, the ANN 36 is configured to determine such inserted length as an accumulation of such incremental changes in successive images of the time sequence of images 35. To do so, the processing device 18 may be configured to determine incremental changes in inserted device length between images of pairs of images of the time sequence of images 35 by inputting each pair of images or features extracted therefrom to the ANN 36 trained to output the incremental change in the inserted length of the instrument, and the processing device 18 adds the determined incremental changes to determine the current inserted length of the instrument. In some embodiments, the determination of the inserted length as the accumulation of incremental changes in inserted length includes inputting the time sequence of images 35 (or features extracted therefrom) to the ANN 36 (implemented as a temporal ANN) trained to output the inserted length based on the inputted time sequence of images or features extracted therefrom.

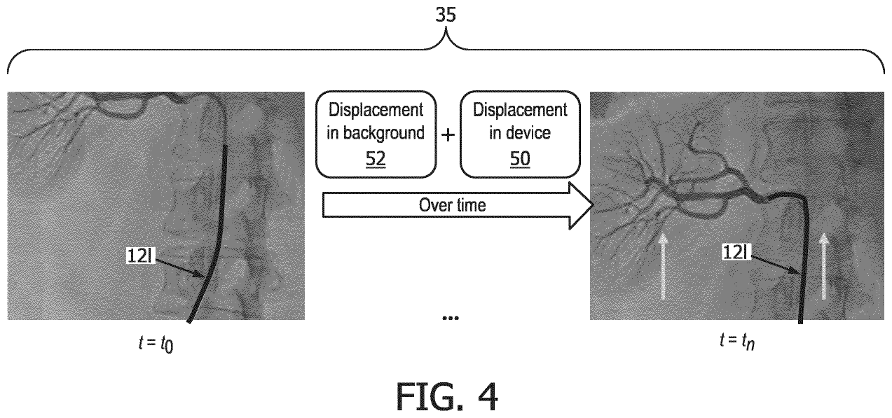

With reference to FIG. 4, an illustrative example of the time sequence of images 35 is shown by way of an illustrative image at time t=t₀ and a later illustrative image at a time t=tₙ. In each image, the depiction of the interventional instrument is indicated and labeled as instrument depiction 12I. FIG. 4 shows that between time t=t₀ and time time t=tₙ the interventional instrument has moved (as seen by comparing its image 12I in the two images), but also the background has moved, as the operator has moved the imaging device 30 to move the imaging field of view (FOV) to capture the branched region. This is indicated in FIG. 4 by a diagrammatic “Displacement in device” 50 and “Displacement in background” 52.

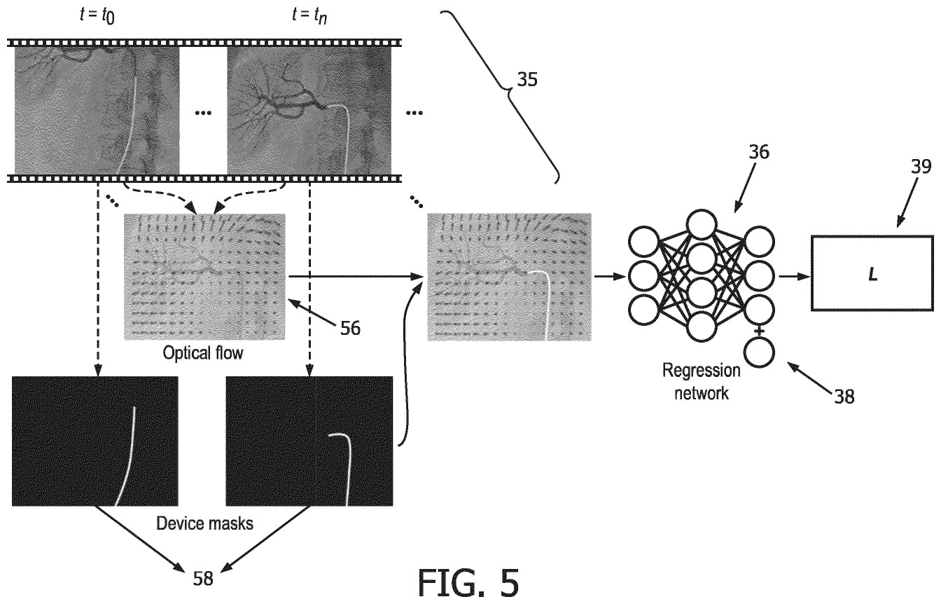

With reference to FIG. 5, in some embodiments, the determination of the currently inserted length of the instrument includes, for each pair of images of a succession of pairs of images of the time sequence of images 35, computing an optical flow field 56 between the images of the pair of images, identifying the portion of the interventional instrument 12 depicted in each image of the pair of images (represented as “Device masks” 58 in FIG. 5), and inputting the optical flow field 56 and the identified portion 58 of the interventional instrument 12 depicted in each image of the pair of images (optionally, along with the additional information 38) to the ANN 36 trained to determine the inserted device length 39 from the inputs. In this approach, the optical flow field 56 captures the background motion or displacement 52 occurring in the time interval between the images of the pair (see FIG. 4), while the identified portions 58 of the interventional instrument 12 depicted in each image captures the device motion or displacement 50 occurring in that time interval. In some embodiments, the computing of the optical flow field 56 includes masking the identified portion of the interventional instrument 12 depicted in each image of the pair of images before computing the optical flow field. Such device masking may ensure that the optical flow field 56 represents only the background motion or displacement 52 and does not have a contribution from the generally independent device motion 50. However, because the fraction of the total area of each image occupied by the (typically thin) interventional instrument 12 is small, in some other embodiments this masking prior to computing the optical flow field 56 may be omitted. In some embodiments, the images of the pair of images may be input directly into the ANN 36 with the optical flow field 56 or identified portion 58 of the interventional instrument 12, allowing the ANN 36 to automatically learn the relevant features that result in accurate inserted device length 39 estimates.

Returning reference to FIG. 2, at an operation 110, the processing device 18 generates a confidence value for the determined inserted length of the interventional instrument. In some embodiments, the confidence value may be estimated directly by the ANN 36 as an additional output. During training of the ANN 36, the confidence (c) output may be compared with the errors (e) (e.g., c=1/e) in length estimation of the interventional instrument 12. Instances that generate lower errors imply high confidence, while instances that generate higher errors imply lower confidence. Alternatively, confidence values may be computed using a dropout layer in the ANN 36. Dropout randomly drops the outputs of a specified number of nodes in the ANN 36, generating a slightly different output for the same input at multiple inference runs. The mean and variance from the multiple outputs can be computed, and as before a smaller variance indicates high confidence (consistent outputs), while a larger variance indicates low confidence (inconsistent outputs). These or other confidence estimation methods learn to associate lower confidence with features that tend to generate higher errors. For instance, ambiguities resulting from the 2D nature of fluoroscopy images, such as foreshortening (i.e., moving out-of-plane), may result in higher errors. Similarly, motion and appearance of background features (e.g., boney landmarks) away from the center of the image may be distorted due to the parallax effect. Parallax effects occurs because the X-ray source 32 is smaller than the X-ray detector 34, meaning that away from the center of the image, the X-ray beams arrive at the detector at a tilted angle. These distortions can result in higher errors.

At an operation 112, the processing device 18 outputs the determined inserted length, for example on the display device 24 in communication with the processing device 18. In some embodiments, a visualization 38 of a length of the interventional instrument 12 is generated and displayed on the display device 24. The estimated length of the interventional instrument 12 is displayed relative to the portion of the anatomy of the patient in which the interventional instrument 12 is inserted.

In some embodiments, the trained ANN 36 may be configured to take as input sequences of fluoroscopy images 35 and other relevant information and to compute the estimated length of the interventional instrument 12 (e.g., guidewire) inserted into the patient body. The ANN 36 may be further configured to compute this estimation by estimating the amount of motion in the background image and the amount of motion in the interventional instrument 12 to estimate the total motion, and to scale this estimate by the size of landmarks visible in the background of the images and/or by the thickness of the interventional instrument 12. This scaling allows the ANN 36 to estimate a metric length of the device inserted into the patient. This estimate may then be used to evaluate the length of a subsequent interventional instrument 12 that will be inserted into the patient. This estimate can be used for downstream evaluations including, but not limited to, estimating the length of interventional instrument 12 that will be subsequently inserted into the patient body. Other downstream evaluations may include identifying anomaly when using the robot 16 to insert the interventional instrument 12. For instance, the robot 16 may keep track of the length of the interventional instrument 12 inserted into the patient at the access site and may compare this known length with the length of the interventional instrument 12 inserted into the patient body. In case of mismatch, the robot 16 may alert the user that the interventional instrument 12 may be, for instance, buckling outside the imaging field of view and the user may take relevant action to resolve the buckling.

To train the ANN 36 retrospective data may be obtained. The retrospective data may be obtained from a large set of historic procedures consisting of (i) sequences of fluoroscopy images containing devices that may be moving, background that may be moving, and devices and background both moving, and (ii) other information about the patient and/or procedure that may be available during the procedure, including but not limited to an amount of C-arm movement, an amount of patient table movement, patient health information (e.g., patient height, patient age, etc.), preoperative or intraoperative 3D image, or any information that allows for the scaling to metric length of an estimated length from fluoroscopy images.

Next, a ground truth length of the interventional instrument 12 inserted into patient body may be obtained from, for example, (i) shape sensed devices (e.g., Fiber Optic RealSense or FORS devices)—the shape and/or other information from these interventional instruments 12 can be used to evaluate which sections of the interventional instrument 12 are in patient body and which are outside; (ii) interventional instruments 12 with an electromagnetic (EM) tracked tip—if the location at which the tip enters the patient body is known and the tip is continuously tracked once the interventional instrument 12 is in patient body, then the length of the interventional instrument 12 in patient body can be evaluated; (iii) the robot 16 in which the length of the interventional instrument 12 that the robot 16 has pushed into the patient body is known, or any manually inserted interventional instrument 12 in which after retrieval of the interventional instrument 12 from patient body, the interventional instrument 12 may be observed to evaluate what section of the interventional instrument 12 was inserted into patient body and the section may be measured; alternatively, the external portion of the interventional instrument 12 closest to the access site may be marked prior to retrieval of the interventional instrument 12 from patient body in order to evaluate what section of the interventional instrument 12 was inserted into patient body and the section may be measured.

As an example, in one implementation, optical flow may be computed between pairs of images in the sequence of fluoroscopy images 35. However, since the interventional instrument 12 is moving independently of the background, it may be segmented out of the optical flow computation. This sequence of optical flow fields with changing masks identifying the interventional instrument 12 may be input into the ANN 36. The ANN 36 may be any architecture that is capable of processing temporal data including but not limited to temporal convolutional networks (TCN), recurrent neural networks (RNN), transformer networks, etc. The ANN 36 uses features from these inputs to evaluate how much of the interventional instrument 12 has been inserted up to scale.

The additional patient information allows the ANN 36 to the scale the estimate to generate a metric length. The additional information 38 is handled differently depending on its type. For instance, PHI (e.g., patient height, age, etc.), amount of C-arm or table movement, or other numerical information may be concatenated into a feature vector (e.g., 1D vector or linear layer a few layers before the output layer) as indicated by a shaded circle in FIG. 5. The ANN 36 trained with fluoroscopy images and patient height, for instance, can learn to associate estimated distances with landmarks seen in the background fluoroscopy image (e.g., vertebra). Another example of additional patient information is 3D image data. 3D image data may be incorporated through registration. If the 2D fluoroscopy and 3D images are registered, then the scale of the anatomy visible in the fluoroscopy images is known.

The ANN 36 may be trained by computing errors (e) between the network output and the ground truth inserted device length. Errors may be computed using any loss function including but not limited to the L1 norm, the L2 norm, negative log likelihood, and so forth. During training, the value of the loss function is typically minimized, and training is terminated when the value of the loss function satisfies a stopping criterion. Sometimes, training is terminated when the value of the loss function satisfies one or more of multiple criteria. Various algorithms have been developed to solve the loss minimization problem including but not limited to Stochastic Gradient Descent “SGD,” batch gradient descent, mini-batch gradient, Adam, and so on. These algorithms compute the derivative of the loss function with respect to the model parameters using the chain rule. This process is called backpropagation since derivatives are computed starting at the last ANN layer or output layer, moving toward the first ANN layer or input layer. These derivatives inform the algorithm how the model parameters must be adjusted in order to minimize the loss function. If the training process is successful, the trained ANN 36 accurately predicts the expected output data from new input data.

The output metric length of the interventional instrument 12 inserted may be visualized on a screen or communicated to the user in another way (e.g., audio feedback). This output informs subsequent steps. For instance, an estimated metric length of a guidewire can help decide the length of catheter to insert over the guidewire, as explained above.

In some embodiments, synthetic data may be used for training where various properties of X-ray image generation can be controlled and, therefore, allow the trained ANN 36 to be more robust. For instance, parallax effects and resulting device distortions may be simulated in order to train the ANN 36 that are robust to distortions in devices away from the center of the image.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A system for determining an inserted length of an interventional instrument the system comprising:
a processor in communication with memory, the processor configured to:
receive imaging data comprising an anatomy within a patient;
identify, from the imaging data, a portion of the interventional instrument inserted into the patient and disposed within the anatomy;
obtain scaling information associated with the imaging data; and
predict the inserted length of the interventional instrument based on the identified portion of the interventional instrument and the scaling information.

2. The system of claim 1, wherein the received imaging data comprises a time sequence of images, and the processor is further configured to predict the inserted length of the interventional instrument as an accumulation of incremental changes in inserted length of the interventional instrument between successive images of the time sequence of images.

3. The system of claim 1, wherein the processor is further configured to:
for each pair of images of a succession of pairs of images of the time sequence of images:
compute an optical flow field between the images of the pair of images;
identifying the portion of the interventional instrument in each image of the pair of images; and
apply a model trained to predict the inserted length based on the optical flow field, the identified portion of the interventional instrument in each image of the pair of images, and the scaling information.

4. The system of claim 3, wherein the processor is configured to mask the identified portion of the interventional instrument in each image of the pair of images before computing the optical flow field.

5. The system of claim 2, wherein the processor is further configured:
apply a model trained to predict the incremental changes in inserted length based on images of pairs of images of the time sequence of images or features extracted therefrom.

6. The system of claim 5, wherein the processor is further configured to:
add the predicted incremental changes to determine the inserted length.

7. The system of claim 1, wherein the processor is configured to:
predict the inserted length of the interventional instrument based on the identified portion of the interventional instrument disposed within the anatomy, the scaling information, and one or more anatomical landmarks of the anatomy in the imaging data.

8. The system of claim 7, wherein the imaging data includes at least two images depicting different views of the portion of the interventional instrument and the one or more anatomical landmarks; and the processor is configured to predict the inserted length of the interventional instrument based on the at least two images depicting the different views.

9. The system of claim 1, wherein the scaling information comprises a size scale associated with the portion of the interventional instrument.

10. The system of claim 1, wherein the scaling information includes one or more of: an amount of movement of a component of an imaging device that acquires the imaging data, patient size information, a three-dimensional (3D) image of the portion of anatomy of a patient in which the interventional instrument is disposed, and information for a dimension of the associated interventional instruments to be inserted into anatomy of the patient.

11. The system of claim 1, wherein the processor is configured to output the predicted inserted length on a display device.

12. The system of claim 11, wherein the processor is further configured to:
generate a visualization of a length of the interventional instrument and
display, on the display device the generated visualization of the length of the interventional instrument.

13. The system of claim 1, wherein the processor is configured to:
generate a confidence value for the inserted length.

14. The system of claim 1, wherein the processor is further configured to:
determine the length from features in a background of images in the imaging data.

15. The system of claim 1, wherein the processor is configured to apply a machine-learning model trained to predict the inserted length of the interventional instrument based on the identified portion of the interventional instrument disposed within the anatomy and the scaling information.

16. The system of claim 1, wherein the imaging data comprises two-dimensional (2D) imaging data of the portion of the interventional instrument and the portion of anatomy of the patient.

17. The system of claim 1, further comprising:
an imaging device configured to acquire the imaging data;
wherein the imaging device is in communication with the processor.

18. A non-transitory computer readable medium having stored a computer program comprising instructions which, when executed by a processor, cause the processor to:
receive imaging data comprising an anatomy within a patient;
identify, from the imaging data, a portion of the interventional instrument inserted into the patient and disposed within the anatomy;
obtain scaling information associated with the imaging data; and
predict the inserted length of the interventional instrument based on the identified portion of the interventional instrument and the scaling information.

19. The non-transitory computer readable medium of claim 18, wherein the instruction, when executed by the processor, further cause the processor to:
for each pair of images of a succession of pairs of images of the time sequence of images:
compute an optical flow field between the images of the pair of images;
identifying the portion of the interventional instrument in each image of the pair of images; and
apply a model trained to predict the inserted length based on the optical flow field and the identified portion of the interventional instrument in each image of the pair of images.

20. A method of determining an inserted length of an interventional instrument, the method comprising:
receiving imaging data comprising an anatomy within a patient;
identifying, from the imaging data, a portion of the interventional instrument inserted into the patient and disposed within the anatomy;
obtaining scaling information associated with the imaging data; and
predicting the inserted length of the interventional instrument based on the identified portion of the interventional instrument and the scaling information.
